(19) United States
(12) Patent Application Publication
(10) Pub. No.: US 2007/0265517 A1
(43) Pub. Date: Nov. 15, 2007

(54) EXPLORATORY TESTING SYSTEM AND ITS APPLICATION TO ABDOMINAL-WALL PUNCTURING

(75) Inventors: Mathias Kraas, Rosssteert (DE); Andreas Muckner, Hamburg (DE); Stephan Klockner, Hamburg (DE)

Correspondence Address: RANKIN, HILL, PORTER & CLARK LLP, 38210 Glenn Avenue, WILLOUGHBY, OH, 44094-7808, US

(73) Assignee: OLYMPUS WINTER & IBE GMBH, Hamburg (DE)

(21) Appl. No.: 11/746,828

(22) Filed: May 10, 2007

(30) Foreign Application Priority Data
May 15, 2006 (DE) 102006022875.8

(51) Int. Cl. A61B 5/04

(52) U.S. Cl. 600/382

(57) ABSTRACT

A surgical exploratory testing system (8, 11) to select a laparoscopic trocar's puncture site (5, 6, 7) through the abdominal wall (1) is characterized in that said system is designed as a coil unit (9) which can be placed on the puncture site (5, 6, 7) and which is connected to an eddy current detector (11).

### BACKGROUND OF THE INVENTION

The present invention relates to a surgical exploratory testing system.

In laparoscopic surgery endoscopic stem implements are inserted through puncture apertures into the abdominal space usually insufflated with CO₂.

After the abdominal space has been insufflated and once said space may be observed internally using an appropriate instrument, further punctures may be carried out devoid of jeopardy. However the first puncture is critical in that the abdominal wall must be pierced without damaging any organs underneath. This constraint entails difficulties even when conventionally palliated by raising the puncture site off the organs underneath it.

Particular problems frequently arise in that the abdominal wall and the organs underneath it knit into each other. As a result the puncture site is always explored beforehand by a suitable means.

Known exploratory systems are puncturing instruments. The known Veress needle is a hollow needle fitted with an internal bar advanced by a spring element. If the Veress needle punctures the abdominal wall and if then the internal bar does advance in proper manner a cavity shall be known to be situated underneath the abdominal wall and as a result a wider trocar spindle may be used at this puncture site. Other known exploratory systems illustratively are designed as trocar spindles with integrated optics. In that case the abdominal wall may be punctured while being optically observed. Following puncturing the abdominal wall, the optics allows for ascertaining whether behind this wall there is a cavity or an adhering organ.

Such known systems are complex, especially in their handling, and frequently entail observational errors.

### BRIEF SUMMARY OF THE INVENTION

The objective of the present invention is to simplify and hence to increase the reliability of such exploratory puncture testing.

This goal is attained in the present invention by the features described in the claims.

It is known to measure eddy currents in body tissue. Applying alternating current (AC) to a coil unit generates an AC magnetic field in turn inducing circular currents about the coil axis in the electrically conducting body tissue that in turn by induction generate back currents in the generating coil or in a separate test coil. Optionally, using different frequencies, a detector element may then detect the tissue conductivity and hence the kind of tissue being tested.

The present invention employs this exploratory testing principle in a system pre-testing the puncture site. A coil system is placed on the puncture site. The connected eddy current test element then provides information about the tissue underneath the coil unit and in particular can determine whether a cavity or an organ is present at the tentative puncture site underneath the abdominal wall. By moving the coil unit to-and-fro, an appropriate puncture site can be determined very simply and rapidly that shall allow for piercing the trocar tube through said abdominal wall in very safe manner.

The design/application of the present invention allows for finding a puncture site in a very simple manner. Such a procedure may be carried out by less skilled personnel than the surgeon, for instance by a technician, when preparing for laparoscopic surgery. Its simplicity and reliability does not require a physician.

In the preferred manner, the coil unit is configured in an electrically insulated manner in a flexurally elastic flat pad. Illustratively, this flat pad is made of an elastomeric material or the like and shall be in the shape of a beer coaster. Accordingly, it can be easily placed on the abdomen and one or more flat coils connected by cables to an eddy current detector may be mounted to it.

Advantageously, the flat pad may be fitted with a central hole centered on the coil axis and hence configured for maximum detection accuracy. Once an appropriate puncture site has been found, said hole may be used to apply a mark to the abdomen or the site may be pierced directly through it.

Preferably, several adjoining coils emitting different signals may also be used. Illustratively, the connected test element then may provide information about a more advantageous puncture site by shifting the coil unit in a given direction along the abdomen.

### BRIEF DESCRIPTION OF THE DRAWINGS

The present invention is shown in illustrative and schematic manner in the appended drawing.

FIG. 1 is a section through the abdomen and the exploratory testing system of the present invention.

FIG. 2 is a topview of the exploratory testing system of FIG. 1, and

FIG. 3 is a topview of another embodiment mode of the exploratory testing system of the present invention.

### DETAILED DESCRIPTION OF THE INVENTION

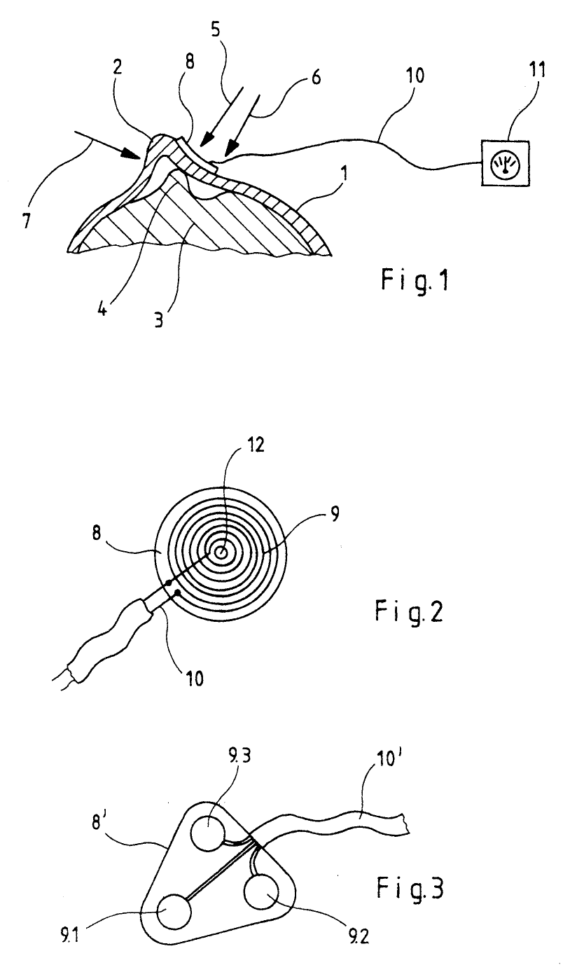

FIG. 1 shows a cross-section of a patient's abdomen and abdominal wall 1, said wall being raised into a fold 2 when preparing a puncture site, for instance using an omitted grip element. Abdominal organs 3 not further differentiated in this Figure are situated underneath the abdominal wall.

FIG. 1 shows that by raising the abdominal wall 1 at the fold 2, said wall is lifted off the abdominal organs 3 at that site except for one spot where the abdominal organs 3 are knitted together with the wall 1.

It is assumed that the first step of a laparoscopic surgery shall be a first puncture for the purpose of insufflating with gas the abdominal space between the abdominal wall 1 and the organs 3 and that thereupon instruments shall be inserted through further punctures.

The first puncture is routinely and tentatively set in the direction of the arrow 5. Now, a check must be run to see whether a puncture at that site is harmless or whether other punctures at the sites of arrows 6 or 7 would be more appropriate.

For that purpose a flat pad 8, shown in topview in FIG. 2, is placed at the intended puncture site 5 on the abdominal wall 1. As shown by FIG. 1, the flat pad is made of a resilient material, for instance an elastomeric material or the like. An electrical coil unit is mounted in an electrically insulating manner inside said flat pad and in a simple embodiment consists of a flat coil 9 as shown in FIG. 2. Said flat coil is connected by two conductors 10 to an eddy current detector 11.

The eddy current detector 11 applies an AC of suitable, for instance variable frequency to the flat coil 9. The generated magnetic field generates eddy currents about the axis of the flat coil 9 in the body tissue situated underneath. The inductive feedback affects the current in the coil 9, and this reaction is detected by the eddy current detector and illustratively may be shown on a display (FIG. 1) of the said eddy current detector.

Regarding the illustrative anatomy shown in FIG. 1, different displays shall result as the flat pad 8 moves from the puncture site 5 to the puncture site 6 or the puncture site 7 because the electrical reaction at the site 5 will be much different due to the local knit 4 of abdominal wall and organ(s) than for the puncture sites 6 and 7 which are situated above a cavity underneath the abdominal wall 1. Accordingly, the surgeon is able to determine very rapidly, by moving the flat pad 8 to-and-fro and by noting the particular displays on the eddy current detector 11, where to safely puncture.

FIG. 3 shows a similar flat pad 8′ fitted with cables 10′ connected to an omitted eddy current detector. The flat pad 8′ comprises three triangularly adjoining coils 9.1, 9.2 and 9.3. As indicated in FIG. 3, said coils each are connected by conductors and the cable 10′ to the eddy current detector. When separately analyzing the outputs of the coils 9.1 through 9.3, a suitable puncture site can be determined, or at least a trend indicating, i.e. that a more advantageous site might lie in the direction of the coil 9.3.

The embodiment mode of the flat pad 8 shown in FIG. 2 comprises a coil which is a transmitting and receiving coil. However, two illustratively mutually concentric coils may be used, one acting as a transmitter and the other as a receiver.

Centrally at the axis of the coil 9, FIG. 2 also shows a hole 12 in said sliding element 8. Once a suitable puncture site has been found using the flat pad 8, a marking may be applied through said hole or puncturing may be carried out directly through it.

1. A surgical exploratory testing system (8, 11) to select a laparoscopic trocar's puncture site (5, 6, 7) through the abdominal wall (1) wherein said exploratory testing system comprises:
a coil unit (9) which can be placed on the puncture site (5, 6, 7) and which is connected to an eddy current detector (11).

2. A method of selecting a laparoscopic trocar's puncture site (5, 6, 7) through an abdominal wall (1) comprising the steps of:
employing a coil unit (9), connected to an eddy current detector (11), as an exploratory testing system; and
placing the coil unit (9) on the puncture site (5, 6, 7) to select the laparoscopic trocar's puncture site (5, 6, 7) through the abdominal wall (1).

3. The system of claim 1, wherein the flat coil unit (9) is configured in a flexurally elastic flat pad (8, 8′) made of an electrically insulating material.

4. The system of claim 3, wherein the flat pad (8) is fitted with a central hole (12).

5. The system of claim 1, wherein the coil unit comprises several mutually adjoining coils (9.1, 9.2, 9.3)

6. The method of claim 2, wherein the flat coil unit (9) is configured in a flexurally elastic flat pad (8, 8′) made of an electrically insulating material.

7. The method of claim 6, wherein the flat pad (8) is fitted with a central hole (12).

8. The method of claim 2, wherein the coil unit comprises several mutually adjoining coils (9.1, 9.2, 9.3)
